(19) United States
(12) Patent Application Publication
(10) Pub. No.: US 2011/0033023 A1
(43) Pub. Date: Feb. 10, 2011

(54) TILT IMAGE SCAN METHOD AND RECONSTRUCTION METHOD AND APPARATUS

(76) Inventor: Ximiao CAO, Beijing (CN)

Correspondence Address: PATRICK W. RASCHE (20459);ARMSTRONG TEASDALE LLP, 7700 Forsyth Boulevard, Suite 1800, St. Louis, MO, 63105, US

(21) Appl. No.: 12/850,299

(22) Filed: Aug. 4, 2010

(30) Foreign Application Priority Data
Aug. 4, 2009 (CN) 200910160292.4

(51) Int. Cl. A61B 6/03

(52) U.S. Cl. 378/4

(57) ABSTRACT

A tilt image scan method includes acquiring reconstruction parameters of a target tilt image, determining on the basis of the reconstruction parameters the minimum beam widths of the rays that should be emitted from the tube at each angle, and controlling the collimator at each angle where the tube locates so that the beam width after the beam passes through the collimator is equal to the corresponding minimum beam width thereby scanning the subject.

### CROSS REFERENCE TO RELATED APPLICATIONS

This application claims the benefit of Chinese Patent Application No. 200910160292.4 filed Aug. 4, 2009, which is hereby incorporated by reference in its entirety.

### BACKGROUND OF THE INVENTION

The present invention relates in general to the Computer Tomography (CT) field, and in particular to a tilt image scan method and reconstruction method and apparatus.

Nowadays, CT is more and more frequently used in the medical field to assist doctors in diagnosing diseases. It can clearly image the organ, bone and blood flow and so on in human body, enabling doctors to see clearly images of the organ, bone and blood flow and so on in human body, thereby performing diagnosis to treat the pathological changes.

Typically, CT usually comprises a gantry that acts as the CT scan section and a table that supports the patient being scanned. Classified by functions, the CT scan section further comprises an X-ray generating section, and a data acquiring section after X-ray passing through the body. As shown in FIG. 8, the gantry comprises: a tube 3 that generates X-ray for scanning; a collimator 4 for limiting the width of the X-ray beam; a detector 5 for receiving X-ray signals and reconstructing tomographic images.

Wherein, the tube 3 usually scans in a circular movement, and obtains a slice of image per rotation. However, human body has inherent physiological curvatures, e.g. the backbone and so on, so there is a need for tilt images. It is well-known to all that X-ray is harmful to human body, thus while a part in the body is imaged with a CT, the dose applied to said part should be as little as possible so long as the image of this part can be obtained. Therefore, in the case of guaranteeing to apply as little dose as possible to the body, how to scan the curvature portion to obtain tilt images is a key research subject in current CT field.

Currently, there are two methods for obtaining tilt images. One is to tilt the gantry, adjust the angle position of the tube and detector relative to the subject, thereby obtaining title images. This method can reduce the dose applied to human body, but the gantry is to be tilted, so that the rotate speed is limited, and the cost is high. The other method is based on image post-processing, i.e. obtaining a set of non-tilt tomographic images by scanning, then processing the obtained set of non-tilt tomographic images by such method as interpolation and so on, thereby acquiring target tilt images. Although this method can guarantee rotation speed and low cost, its scan range is large, so the dose applied to human body is large. Thus, this method is not advantage to human health.

### BRIEF DESCRIPTION OF THE INVENTION

The main technical problem to be solved by the present invention is to provide a tilt image scan method and reconstruction method and apparatus with low-dose and low-cost.

In one aspect, a tilt image scan method is provided in which the subjected may be scanned by X-ray emitted from a tube through a collimator. The tilt image scan method includes acquiring the reconstruction parameters of a target tilt image, determining, on the basis of said reconstruction parameters, the minimum beam widths of the rays that should be emitted from the tube at each angle, and controlling the collimator at each angle where the tube locates so that the beam width after the beam passes through the collimator is equal to the corresponding minimum beam width, thereby scanning the subject.

The step of acquiring reconstruction parameters of a target tilt image includes scanning the subject to obtain its 90 degree and 0 degree location images, and determining, on the basis of said location images, the reconstruction parameters of the target tilt image, wherein the reconstruction parameters include position, angle, layer thickness and size of reconstruction field of view.

Furthermore, the step of determining on the basis of said reconstruction parameters the minimum beam widths of rays that should be emitted from the tube at each angle includes obtaining, on the basis of said position, angle and thickness as well as required resolution, the layer thickness of a non-tilt tomographic image, obtaining, on the basis of said layer thickness and said angle, the scan range of the non-tilt tomographic images necessary for reconstructing the target tilt image, obtaining, on the basis of the position and angle of said target tilt image, the reconstruction areas on each slice of non-tilt tomographic images for reconstructing said target tilt image, and determining, on the basis of said reconstruction area, the minimum beam widths of said tube at each angle.

In another aspect, a tilt image reconstruction method includes acquiring the reconstruction parameters of a target tilt image, determining, on the basis of said reconstruction parameters, the minimum beam widths of the rays that should be emitted from the tube at each angle, controlling the collimator at each angle where the tube locates, so that the beam width after the beam passes through the collimator is equal to the corresponding minimum beam width, thereby scanning the subject, and reconstructing the target tilt image on the basis of the data obtained from the above scan.

The step of acquiring reconstruction parameters of a target tilt image includes scanning the subject to obtain its 90 degree and 0 degree location images, and determining, on the basis of said location images, the reconstruction parameters of the target tilt image, wherein the reconstruction parameters include position, angle, layer thickness, and size of reconstruction field of view.

Furthermore, the step of determining on the basis of said reconstruction parameters the minimum beam widths of the ray that should be emitted from the tube at each angle includes obtaining, on the basis of said position, angle and thickness as well as required resolution, the layer thickness of a non-tilt tomographic image, obtaining, on the basis of said layer thickness and said angle, the scan range of the non-tilt tomographic images necessary for reconstructing the target tilt image, obtaining, on the basis of the position and angle of said target tilt image, the reconstruction areas on each slice of non-tilt tomographic images for reconstructing said target tilt image, and determining, on the basis of said reconstruction area, the minimum beam widths of said tube at each angle.

Said reconstructing the target tilt image on the basis of the data obtained from the above scan further comprises reconstructing by a local reconstruction technique or reconstruct by filtered back-projection.

In another aspect, a tilt image scan apparatus includes an acquisition unit for acquiring the reconstruction parameters of a target tilt image, a first unit for determining, on the basis of said reconstruction parameters, the minimum beam widths of the rays that should be emitted from the tube at each angle, and a control unit for controlling the collimator at each angle where the tube locates so that the beam width after the beam passes through the collimator is equal to the corresponding minimum beam width, thereby scanning the subject.

Further, the tilt image scan apparatus according to the present invention further comprises a unit for reconstructing the target tilt image on the basis of the data obtained from the above scan.

The acquisition unit for acquiring the reconstruction parameters of a target tilt image includes a unit for scanning the subject to obtain its 90 degree and 0 degree location images, and a second unit for determining, on the basis of said location images, the reconstruction parameters of the target tilt image, wherein the reconstruction parameters include position, angle, layer thickness and size of reconstruction field of view.

Furthermore, the first unit for determining on the basis of said reconstruction parameters the minimum beam widths of the rays that should be emitted from the tube at each angle includes a first unit for obtaining, on the basis of said position, angle and thickness as well as required resolution, the layer thickness of a non-tilt tomographic image, a second unit for obtaining, on the basis of said layer thickness and said angle, the scan range of the non-tilt tomographic images necessary for reconstructing the target tilt image, and for obtaining, on the basis of the position and angle of said target tilt image, the reconstruction areas on each slice of non-tilt tomographic images for reconstructing said target tilt image, and a third unit for determining, on the basis of said reconstruction areas, the minimum beam widths of said tube at each angle.

Wherein, the unit for reconstructing the target tilt image on the basis of the data obtained from the above scan further comprises the unit for reconstructing the target tilt image on the basis of the data obtained from the above scan further comprises a unit for reconstructing by a local reconstruction technique or reconstructing by filtered back-projection.

The present invention determines the minimum beam widths of the ray that should be emitted from the tube at each angle on the basis of the reconstruction parameters necessary for reconstructing the target tilt image, then the collimator is controlled at each angle where the tube locates such that the beam width after the beam passes through the collimator is equal to the corresponding minimum beam width to scan the subject, so that the subject is scanned and the tilt image is obtained under the condition that the gantry does not have to be tilted and low dose is guaranteed. Therefore, the technical solution of the present invention is cost-efficient, and can guarantee not to add useless dose due to large scan range.

### BRIEF DESCRIPTION OF THE DRAWINGS

The technical solution of the present invention is described by means of embodiments in combination with the following figures so that the disclosure of the present invention can be understood more thoroughly:

FIG. 1 is a flowchart of the tilt image scan method according to the present invention;

FIG. 2 is a schematic diagram of the target area on the non-tilt tomographic image according to the present invention;

FIG. 3 is a schematic diagram of the positional relationship between the target tilt image and the non-tilt tomographic image according to the present invention;

FIG. 4 is a schematic diagram of the geometrical relation for solving the ray beam width of the target area according to the present invention

FIG. 5 is a schematic diagram of classification of the area where the tube locates according to the present invention;

FIG. 6 is another schematic diagram of classification of the area where the tube locates according to the present invention;

FIG. 7 is still another schematic diagram of classification of the area where the tube locates according to the present invention;

FIG. 8 is a structural schematic diagram of the CT scan section in CT;

FIG. 9 is a flowchart of the tilt image reconstruction method according to the present invention;

FIG. 10 is a flowchart of the subdivision of step 1 in FIG. 1.

### DETAILED DESCRIPTION OF THE INVENTION

The specific embodiments of the present invention are described in details below, but the present invention is not limited to the following embodiments.

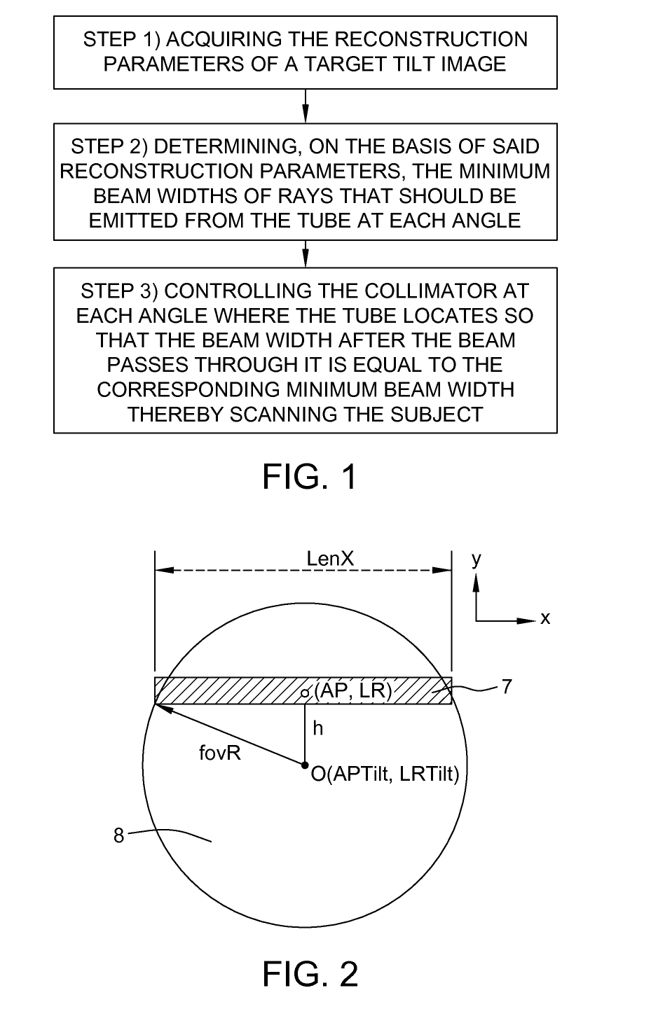

FIG. 1 shows a tilt image scan method the subject is scanned by X-ray emitted from a tube through a collimator. The method includes acquiring the reconstruction parameters of a target tilt image, determining, on the basis of said reconstruction parameters, the minimum beam widths of the X-rays that should be emitted from the tube at each angle, and controlling the collimator at each angle where the tube locates so that the beam width after the beam passes through the collimator is equal to the corresponding minimum beam width, thereby scanning the subject.

As for the step 1), there are a plurality of ways to acquire the reconstruction parameters.

For example, as shown in FIG. 10, the following two steps can be adopted to acquire the reconstruction parameters. The subject can be scanned to obtain its 90 degree and 0 degree location images, and the reconstruction parameters of the target tilt image can be determining based on the location images.

The steps can also be carried out by means of an external locating device, e.g. perform coarse positioning by a position lamp added on the CT scanner, and specific reconstruction parameters may be given out in virtue of the background knowledge of the operator, and so on.

Wherein, the reconstruction parameters may include position, angle, layer thickness and size of reconstruction field of view.

In addition, the reconstruction parameters can be determined by obtaining, on the basis of said position, angle and thickness as well as required resolution, the layer thickness of a non-tilt tomographic image, obtaining, on the basis of said layer thickness and said angle, the scan range of the non-tilt tomographic images necessary for reconstructing the target tilt image, obtaining, on the basis of the position and angle of said target tilt image, the reconstruction area on each slice of non-tilt tomographic image for reconstructing said target tilt image, and determining, on the basis of said reconstruction area, the minimum beam widths of said tube at each angle.

It can be seen from the above that the tilt image scan method of the present invention first scans the subject to obtain 90 degree and 0 degree location images, then determines, on the basis of said location images, the reconstruction parameters of the target tilt image (which can also be determined by the user), such as position, angle, layer thickness and size of reconstruction field of view, wherein, first a physical coordinate system is defined, the direction of the cradle going in and out is the Z-direction, the gantry being non-tilt means that the plane where the tube 3 and detector 5 locate is the X-Y scan plane. Wherein, the direction parallel with the ground is the X-direction, the direction perpendicular to the ground is the Y-direction. The position of the target tilt image means the coordinate positions in the three directions (X, Y, Z) within said coordinate system; the angle means the included angle between the target tilt image and the X-Y plane; the layer thickness means the vertical thickness of the plane where the target tilt image locates; the size of reconstruction field of view means the size of the target area that the user is interested in within the plane where the target tilt image locates; the reconstruction field of view is in general a circular area, and the size of the reconstruction field of view is usually described by means of the radius of the circle.

Next, determining the minimum beam widths of the ray that should be emitted from the tube at each angle on the basis of said position, angle, layer thickness and size of reconstruction field of view, and in the end, controlling the collimator at each angle where the tube locates so that the beam width after the beam passes through the collimator is equal to the corresponding minimum beam width, thereby scanning the subject. In this way, the dose incident on the subject can be minimized, thus reducing the harm of X-ray to the subject.

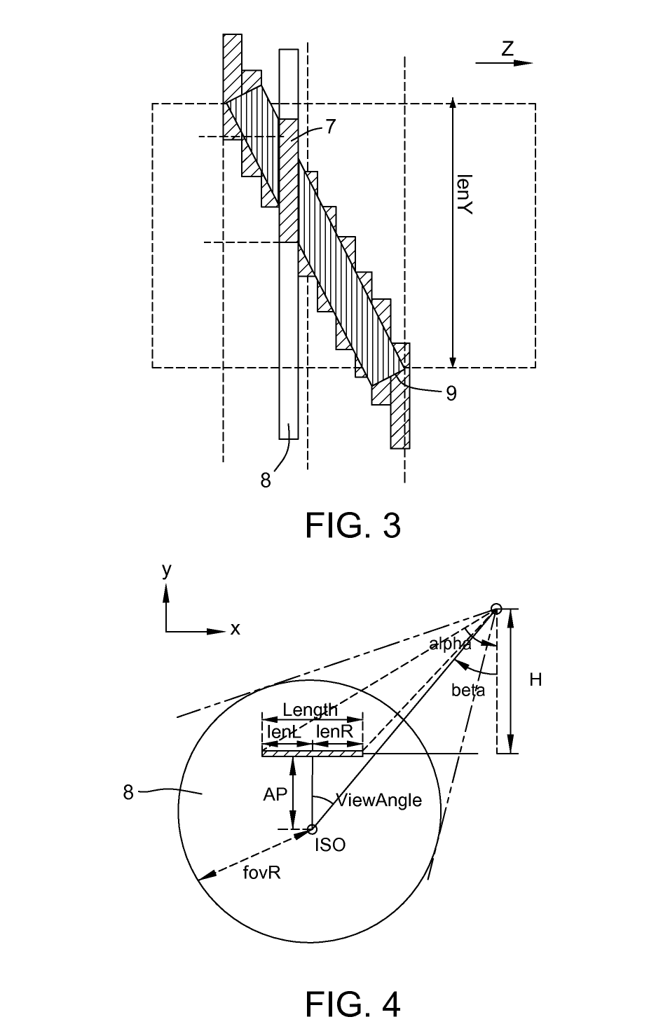

As shown in FIG. 2 and FIG. 3, FIG. 2 illustrates a schematic diagram of the target area 7 (also called the overlapping section of the non-tilt tomographic image and the target tilt image) on the non-tilt tomographic image 8, the point (APTilt, LRTilt) is the reconstruction center O of the non-tilt tomographic image 8, the radius is fovR, the distance from the reconstruction center O to the target area 7 is h. FIG. 3 shows in the direction of the Z axis the positional relationship between the target tilt image 9 and the non-tilt tomographic image 8, then the reconstruction area length LenX in the X-direction within the non-tilt tomographic image and the reconstruction area length LenY in the Y-direction within the non-tilt tomographic image are:

$$LenX = \{ \begin{matrix}\sqrt{{fovR}^{2} - {( fovR - i * thicknessOfNoneTilt )}^{2}} \\ if fovR - ( i + 0.5 ) * thicknessOfNoneTilt > 0 \\ \sqrt{{fovR}^{2} - {( fovR - ( i + 1 ) * thicknessOfNoneTilt )}^{2}} \\ else\end{matrix} \\ LenY = \{ \begin{matrix}\frac{thicknessOfNoneTilt * cos ( AngleTilt ) + thicknessOfTilt}{sin ( AngleTilt )} & \\ \begin{matrix}if \frac{thicknessOfTilt}{sin ( AngleTilt )} < 2 * fovR & \\ 2 * fovR & else\end{matrix} & \end{matrix} \qquad$$

Wherein, thicknessOfNoneTilt represents the thickness of the non-tilt tomographic image; AngleTilt represents the inclination angle of the target tilt image; thicknessOfTilt represents the thickness of the target tilt image; fovR represents the radius of the reconstruction field of view.

On the basis of such known information as the target tilt image's position, inclination angle, reconstruction field of view and layer thickness and so on, the reconstruction area on each non-tilt tomographic image necessary for reconstructing the tilt image is calculated.

The ray beam width is limited according to the reconstruction area of each slice of non-tilt tomographic image. The included angle from the X-ray at the two ends of the beam area to the line connecting the tube and the mechanical center of rotation represents the beam width to which each tube position corresponds. As shown in FIG. 4, it is supposed that the included angles of the rays at two ends of the ray beam are respectively alpha and beta, the distance from the tube to the geometrical center of rotation ISO is Tube2ISO; the included angle of the tube 3 and the 12 o'clock position (the initial position of the tube 3, its ViewAngle=0; ViewAngle means the rotation angle of the tube 3 relative to the initial position during the rotation of the tube 3) is ViewAngle. With the geometrical center of rotation ISO being the origin, the 3 o'clock direction (see the direction of 3 o'clock in a watch) being the positive direction, then on the non-tilt tomographic image, the coordinate of the center of the rectangle area 7 under said coordinate system is (LR, AP), wherein AP is the distance from the rectangle center to the geometrical center of rotation in the Y-direction, LR is the distance from the rectangle center to the geometrical center of rotation in the X-direction. Besides, as for the size of the rectangle area 7, the coverage length in the X-direction is LenX, the coverage length in the Y-direction is LenY. Therefore, the target problem becomes the calculation of a minimum beam width necessary for reconstructing said rectangle area.

Firstly, the problem is simplified to be that, at some particular tube position (angle is ViewAngle), the target area 7 (which means the non-tilt tomographic image necessary for reconstructing the target tilt image, also called an overlapping section of the non-tilt image and the tilt image) is a ray width required by a segment parallel with the X-direction.

Wherein, the vertical distance from the tube 3 to the segment 7 is: multiplying the distance from the tube 3 to the geometrical center of rotation ISO by the cosine of the tube angle, and then subtracting the distance from the segment to the geometrical center of rotation ISO.

Secondly, by trigonometric function, calculating the included angle from left end of the target segment and the tube 3 to the vertical line of the segment and the included angle from right end of the target segment to the vertical line respectively.

In the end, through ViewAngle, a right triangle is formed by the tube 3, segment end, and the vertical point of the tube 3 to the straight line where the segment belongs to, and in this triangle, by tangent trigonometric function relation, calculating the included angle from the left and right ends of the segment to the line connecting the tube 3 and the center of rotation.

The whole calculation process is as follows:

lenL=length/2−LR,lenR=Length/2+LR;

H=Tube2ISO*cos(viewAngle)−AP;

len1=lenL+AP*tan(viewAngle);

len2=lenR+AP*tan(viewAngle);

alpha=arctan((len1+H*tan(viewAngle))/H);

beta=arctan((len2+H*tan(viewAngle))/H);

gama=abs(alpha+beta);

Lessdose=(1−gama/xrayAngle)*100%

Wherein, gama is the angle width of the ray beam; Lessdose is the percentage of dose reduction as compared with regular scan manner.

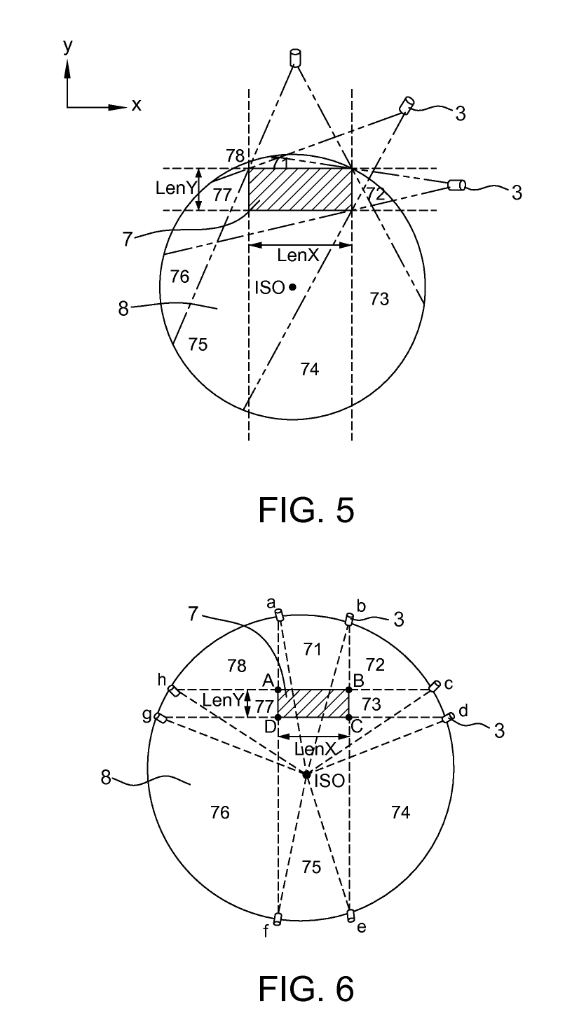

With respect to the situation where the target reconstruction area is a rectangle, simplification can be made according to the special area where the ViewAngle is located to form a series of above problems. As shown in FIG. 5, ViewAngle is divided into 8 areas (i.e. rotating the tube one round and 8 specific ViewAngle areas is divided), i.e. top left area 78, top area 71, top right area 72, left area 77, right area 73, bottom left area 76, bottom area 75, bottom right area 74 of the target rectangle area 7. The target area 7 in this example is a rectangle, whose length and width are LenX and LenY respectively, and the length LenXY of the rectangle diagonal.

Now determining the area where the radiation source locates according to said target area 7.

As shown in FIG. 6, first calculating the coordinates of the four vertexes of the rectangle area (the target area) 7:

A(LR−LenX/2,AP+LenY/2)

B(LR+LenX/2,AP+LenY/2)

C(LR+LenX/2,AP−LenY/2)

D(LR−LenX/2,AP−LenY/2)

Then, by the coordinates of the four vertexes and the geometrical triangle, calculating the tube positions (ViewAngle) to which the 8 points a, b, c, d, e, f, g, h correspond:

ViewAngleₐ=arcsin(Ax/R)=arcsin((2LR−LenX)/2R)

ViewAngle_{b}=arcsin(Bx/R)=arcsin((2LRz,900 LenX)/2R)

ViewAngle_{c}=arccos(By/R)=arcsin((2AP+LenY)/2R)

ViewAngle_{d}=arccos(Cy/R)=arcsin((2AP−LenY)/2R)

ViewAngleₑ=π−ViewAngle_{b}

ViewAngle_{f}=π−ViewAngleₐ

ViewAngle_{g}=2π−ViewAngle_{d}

ViewAngleₕ=2π−ViewAngleₑ

Wherein, the tube positions between a to b, or between e to f, belong to the top area 71 or bottom area 75; the tube positions between c to d, or between g to h, belong to the right area 73 or left area 77; other tube positions belong to the top left area 78 or bottom left area 76 or top right area 72 or bottom right area 74.

The width of the object to be covered by ray can be obtained according to the area where the tube locates:

$$Length = \{ \begin{matrix}LenX & when bulb 3 is in area 71 or 75 \\ \sqrt{( {LenY}^{2} + {LenX}^{2} )} & when bulb 3 is in area 78 or 76 or 72 or 74 \\ LenY & when bulb 3 is in area 73 or 77\end{matrix}$$

When the tube is in the top area 71 or in the bottom area 75, the problem of the target area being a rectangle can be transformed into a problem of the target area being a segment whose length is LenX. Likewise, when the tube is in the top left area 78 or bottom left area 76 or top right area 72 or bottom right area 74, the above problem can be transformed into a problem of a segment whose length is rectangle diagonal length; when the tube is in the right area 73 or left area 77, the above problem can be transformed into a problem of a segment whose length is LenY.

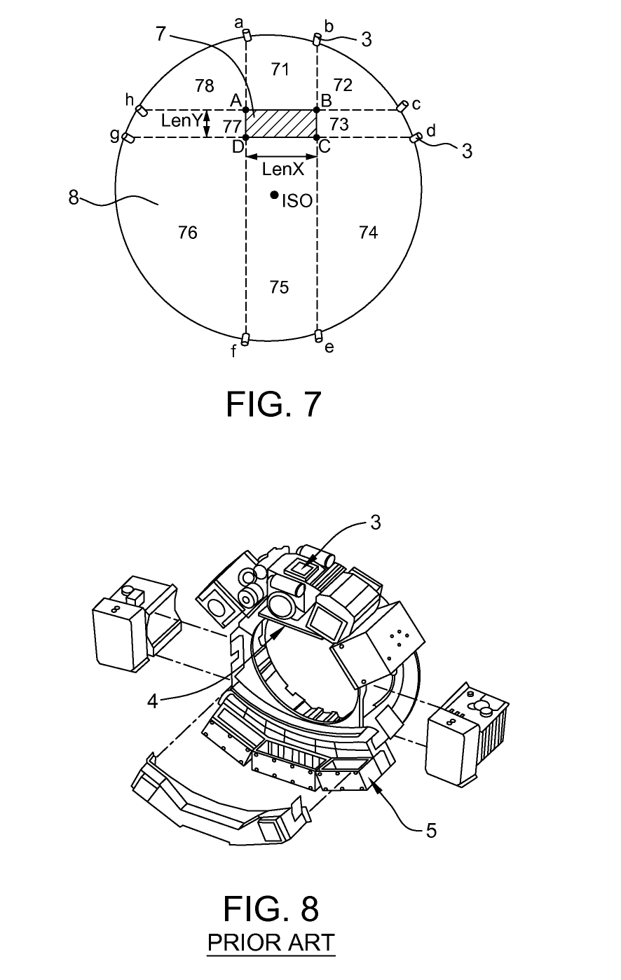

As shown in FIG. 7, which is a schematic diagram of classification of the area where the tube locates. According to the position of the target rectangle 7, the trail of the tube 3 is divided into three situations.

Situation 1: the beam width can be determined by the segment length of the horizontal side of the rectangle, i.e. the rectangle calculation problem can be transformed into a problem of horizontal segment.

Situation 2: the beam width can be calculated by the diagonal segment of the rectangle, i.e. the problem of rectangle calculation can be transformed into a problem of diagonal segment.

Situation 3: the beam width can be determined by the segment length of the vertical side of the rectangle, i.e. the problem of rectangle calculation can be transformed into a problem of vertical segment.

In addition, by performing corresponding transformation on the coordinate, the rectangle target area problem can be transformed into a series of segment target area problems. This is because that the precondition of the above calculation for the target area 7 being a segment is: the target segment is parallel with the X-direction. The above situation 1 accords with this assumption. As for the situation 2 and situation 3, the target segment and the X-direction have a known included angle. The deduction process of said situations is similar to that of the situation 1. Here are two methods: one is to adjust the initial position of ViewAngle to turn the situation having an angle into a situation of parallel, the other is to turn the target segment length having an angle into an equivalent parallel segment length.

In summary, to handle the problem of computing the minimum ray beam of a known rectangle area 7, first the tube 3 position is divided into 8 areas according to the position of the rectangle, and with respect to each area, coordinate transformation is performed, the problem is transformed into computing the beam width to which the segment parallel with the X-direction corresponds, and on the basis of the derived formula, the corresponding minimum X-ray beam widths at each angle in the area are computed. 8 areas are integrated to obtain the X-ray beam width of each tube sample angle necessary for reconstructing the rectangle area 7, thus obtaining the beam width scanning said rectangle area.

For the reconstruction of a target tilt image, rectangle area reconstructions inside multiple slices of non-tilt tomographic images are required. That the projection data covering the rectangle area in each acquired slice of non-tilt tomographic image can ensure the reconstruction of the rectangle area, thereby ensuring the reconstruction of the target tilt image.

For the same reason, the beam in the Z-direction can be limited. The computing process is the same as stated above.

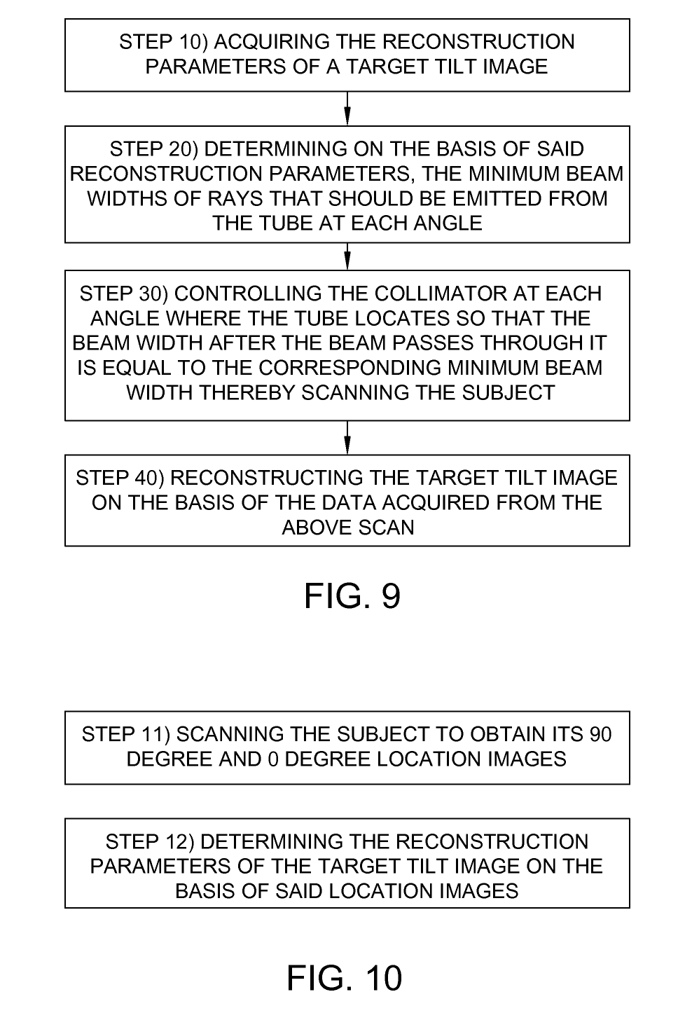

As shown in FIG. 9, the present invention further discloses a tilt image reconstruction method, the subject may be scanned by X-ray emitted from a tube through a collimator. The method includes acquiring 10 the reconstruction parameters of a target tilt image, determining 20, on the basis of said reconstruction parameters, the minimum beam width of the ray that should be emitted from the tube at each angle, controlling 30 the collimator at each angle where the tube locates so that the beam width after the beam passes through the collimator is equal to the corresponding minimum beam width, thereby scanning the subject, and reconstructing 40 the target tilt image on the basis of the data acquired from the above scan.

Further, the step 10 may be achieved by scanning 101 the subject to obtain its 90 degree and 0 degree location images, and determining 102, on the basis of said location images, the reconstruction parameters of the target tilt image. Surely, the step 10) can also be achieved by other manners, e.g. using an external locating device, human eyes, and so on. For example, coarse positioning can be performed by a position lamp added on the CT scanner, and reconstruction parameters can be given out in virtue of the background knowledge of the operator, and so on.

Wherein, the reconstruction parameters include position, angle, layer thickness and size of reconstruction field of view.

In addition, the step 20 includes obtaining 201, on the basis of said position, angle and thickness as well as required resolution, the layer thickness of a non-tilt tomographic image, obtaining 202, on the basis of said layer thickness and said angle, the scan range of the non-tilt tomographic image necessary for reconstructing the target tilt image, and obtaining, on the basis of the position and angle of said target tilt image, the reconstruction areas on each slice of non-tilt tomographic image for reconstructing said target tilt image, and determining 203, on the basis of said reconstruction area, the minimum beam widths of said tube at each angle.

Further, the step 40) can perform reconstruction by a local reconstruction technique or filtered back-projection (FBP).

In additional to step 40), other steps are identical with those in the tilt image scan method, and will not be discussed further.

At present, a lot of methods can be used for local reconstruction, e.g. projection space based back-projection filter (BPF) or projection onto convex sets (POCS) iterative reconstruction and so on. These methods can directly reconstruct the pixel image information on the target tilt image. Take the projection space based image post-processing method as an example: first the target rectangle area of each slice of non-tilt tomographic image can be acquired by an improved FBP algorithm, and then the image pixel information on the tilt image can be obtained by interpolation algorithm. Specific interpolation algorithms include linear interpolation algorithm, Lagrange interpolation algorithm, spline interpolation algorithm, and so on.

Correspondingly, the present invention further discloses a tilt image scan apparatus that includes an acquisition unit for acquiring the reconstruction parameters of a target tilt image, a first unit for determining the minimum beam widths of the rays that should be emitted from the tube at each angle, a control unit for controlling the collimator at each angle where the tube locates so that the beam width after the beam passes through the collimator is equal to the corresponding minimum beam width, thereby scanning the subject, and a reconstruction unit for reconstructing the target tilt image on the basis of the data obtained from the above scan.

Further, the acquisition unit for acquiring the reconstruction parameters of a target tilt image includes a scanning unit for scanning the subject to obtain its 90 degree and 0 degree location images, and a second unit for determining, on the basis of said location images, the reconstruction parameters of the target tilt image, wherein the reconstruction parameters include position, angle, layer thickness, and size of reconstruction field of view.

In addition, the first unit for determining on the basis of said reconstruction parameters the minimum beam widths of the rays that should be emitted from the tube at each angle includes a first unit for obtaining, on the basis of said position, angle and thickness as well as required resolution, the layer thickness of a non-tilt tomographic image, a second unit for obtaining, on the basis of said layer thickness and said angle, the scan range of the non-tilt tomographic image necessary for reconstructing the target tilt image, and for obtaining, on the basis of the position and angle of said target tilt image, the reconstruction areas on each slice of non-tilt tomographic image for reconstructing said target tilt image, and a third unit for determining, on the basis of said reconstruction areas, the minimum beam widths of said tube at each angle.

Wherein, the unit for reconstructing the target tilt image on the basis of the data obtained from the above scan further comprises a unit for reconstructing by a local reconstruction technique or reconstructing by filtered back-projection.

Now, the technical solution of the present invention is adopted to acquire tilt images of human abdomen. Detailed description is as follows:

First, scanning the abdomen to obtain its 90 degree and 0 degree location images;

Then, determining, on the basis of said location images, the reconstruction parameters of the tilt images of the abdomen:

Tilt angle (AngleTilt)=15 degree

Scan field of view (SFOV)=43 cm

Reconstruction field of view (DFOV)=20 cm

Tilt layer thickness (thicknessOfTilt)=3 mm

Reconstruction center O coordinate (APTilt, LRTilt) (0, 0), wherein the reconstruction center is the coordinate position corresponding to the geometrical center of rotation ISO.

Next, determining on the basis of the reconstruction parameters the minimum beam width of the ray that should be emitted from the tube at each angle:

A. Adaptively selecting the layer thickness of the non-tilt tomographic image according to the relation of the angle and the tilt layer thickness. The layer thickness of the non-tilt tomographic images (thicknessOfNoneTilt) selected in this instance is 2 mm.

B. Computing the scan ranges of each slice of non-tilt tomographic images.

$$\begin{matrix}imageNumNoneTilt = [ DFOV * sin ( AngleTilt ) + \\ TiltThickness / cos ( AngleTilt ) ] / \\ NoneTiltThickness \\ = [ 200 * sin ( 15 ° ) + 3 / cos ( 15 ° ) ] / 2 \\ = ⌈ 27.43 ⌉ \\ = 28\end{matrix}$$

Therefore, 28 slices of non-tilt tomographic images need to be reconstructed. Now computing the reconstruction area for each slice of non-tilt tomographic images.

For the first slice of non-tilt image (NoneTiltID=1), the reconstruction area is computed as follows:

$$\begin{matrix}{LenY}_{1} = \frac{thicknessOfNoneTilt * cos ( Angletilt ) + thicknessOfTilt}{sin ( AngleTilt )} \\ = \frac{2 * cos ( 15 ° ) + 3}{sin ( 15 ° )} \\ = 19.0552 mm\end{matrix}$$

$$\begin{matrix}{LenX}_{1} = 2 * \sqrt{{fovR}^{2} - {( fovR - NoneTiltID * thicknessOfNonetilt )}^{2}} \\ = 2 * \sqrt{{100}^{2} - {( 100 - 1 * 2 )}^{2}} \\ = 39.7995 mm\end{matrix}$$

For the second slice of non-tilt tomographic image (NoneTiltID=2), the reconstruction area is computed as follows:

$$\begin{matrix}{LenY}_{2} = \frac{thicknessOfNoneTilt * cos ( Angletilt + thicknessOfTilt )}{sin ( AngleTilt )} \\ = \frac{2 * cos ( 15 ° ) + 3}{sin ( 15 ° )} \\ = 19.0552 mm\end{matrix}$$

$$\begin{matrix}{LenX}_{2} = 2 * \sqrt{{fovR}^{2} - {( fovR - NoneTiltID * thicknessOfNonetilt )}^{2}} \\ = 2 * \sqrt{{100}^{2} - {( 100 - 2 * 2 )}^{2}} \\ = 56 mm\end{matrix}$$

The computing of reconstruction area for other non-tilt tomographic images is done in the same manner.

C. Computing the position information of the required area on each slice of non-tilt tomographic images.

For the first slice of non-tilt tomographic image (NoneTiltID=1), the position information of the required area thereon is:

AP₁=APTilt+fovR*cos(tiltAngle)/imageNumNoneTilt*(imageNumNoneTilt−NoneTiltID)=0+100*cos(15°)/28*(28−1)=93.1428LR₁=LRTilt=0

For the second slice of non-tilt tomographic image (NoneTiltID=2), the position information of the required area thereon is:

AP₂=APTilt+fovR*cos(tiltAngle)/imageNumNoneTilt*(imageNumNoneTilt−NoneTiltID)=0+100*cos(15°)/28*(28−2)=89.6931 mm LR₂=LRTilt=0 mm

The position information of the required area on every slice of non-tilt tomographic images is computed in the same manner.

In the reconstruction of a target tilt image, projection data of different angles (ViewAngle) shall be collected for reconstruction. From the scanned geometrical structures, the distance from the tube to the center of reconstruction can be obtained (Tube2ISO=900 mm)

As for the first non-tilt tomographic image:

LR=LR₁=0 mm

AP=AP₁=93.1428 mm

LenY=LenY₁=19.0552 mm

LenX=LenX₁=39.7995 mm

First computing the corresponding ViewAngles of 8 areas:

$$\begin{matrix}{viewAngle}_{a} = arc sin ( \frac{2 * LR - LenX}{2 * Tube 2 ISO} ) \\ = arcsin ( \frac{2 * 0 - 39.7995}{2 * 900} ) \\ = - 1.2670 °\end{matrix}$$

$$\begin{matrix}{viewAngle}_{b} = arcsin ( \frac{2 * LR + LenX}{2 * Tube 2 ISO} ) \\ = arcsin ( \frac{2 * 0 + 39.7995}{2 * 900} ) \\ = 1.2670 °\end{matrix}$$

$$\begin{matrix}{viewAngle}_{c} = arcsin ( \frac{2 * AP + LenY}{2 * Tube 2 ISO} ) \\ = arcsin ( \frac{2 * 93.1428 + 19.0552}{2 * 900} ) \\ = 83.4495 °\end{matrix}$$

$$\begin{matrix}{viewAngle}_{d} = arcsin ( \frac{2 * AP - LenY}{2 * Tube 2 ISO} ) \\ = arcsin ( \frac{2 * 93.1428 + 19.0552}{2 * 900} ) \\ = 84.6692 °\end{matrix}$$

$${viewAngle}_{e} = 180 ° - {ViewAngle}_{b} = 180 - 1.2670 = 178.7330 °$$

$${viewAngle}_{f} = 180 ° - {ViewAngle}_{a} = 180 - ( - 1.2670 ) = 181.2670 °$$

$${viewAngle}_{g} = 360 ° - {ViewAngle}_{d} = 360 - 84.6692 = 275.3308 °$$

$${viewAngle}_{h} = 360 ° - {ViewAngle}_{c} = 360 - 83.4495 = 276.5505 °$$

Now take ViewAngle=0° as an example:

Θ viewAngleε[viewAngleₐ,viewAngle_{b}], the tube is located in the top area 71 and bottom area 75

$$∴ Length = lenX = 39.7995 mm ;$$

$$lenL = Length / 2 - LR = 39.7995 / 2 - 0 = 19.8998$$

$$lenR = Length / 2 + LR = 39.7995 / 2 + 0 = 19.8998$$

$$H = Tube 2 ISO * cos ( viewAngle ) - AP = 900 * cos ( 0 ° ) - 0 = 900$$

$$\begin{matrix}len 1 = lenL + {AP}_{1} * tan ( viewAngle ) \\ = 19.8995 + 93.1428 * tan ( 0 ° ) \\ = 19.8998\end{matrix}$$

$$\begin{matrix}len 2 = lenR - {AP}_{1} * tan ( viewAngle ) \\ = 19.8998 + 93.1428 * tan ( 0 ° ) \\ 19.8998\end{matrix}$$

$$\begin{matrix}alpha = act tan ( \frac{len 1 + H * tan ( viewAngle )}{H} ) \\ = act tan ( \frac{19.8998 + 900 * tan ( 0 )}{900} ) \\ = 1.2667 °\end{matrix}$$

$$\begin{matrix}beta = act tan ( \frac{len 2 + H * tan ( viewAngle )}{H} ) \\ = act tan ( \frac{19.8998 + 900 * tan ( 0 )}{900} ) \\ = 1.2667 °\end{matrix}$$

$$gama = abs ( alpha + gama ) = abs ( 1.2667 + 1.2667 ) = 2.5334 °$$

The area of ray can be determined through alpha and beta, gama is the total width of ray.

The minimum beam width of individual ViewAngles can be derived by similar computing process.

In summary, the X-ray radiation dose can be reduced by limiting the minimum beam width of each ViewAngle. Compared with the method without beam limitation, the dose can be reduced by 74%.

In the end, the acquired projection data are reconstructed to obtain a target tilt image and that is displayed to the user.

Although the specific embodiments of the present invention have been described in combination with figures, a person having ordinary skill in the art can make various modification, amendment and equivalent substitution without departing from the spirit and scope of the present invention, and such modification, amendment and equivalent substitution shall fall within the spirit and scope defined by the attached claims.

1. A tilt image scan for scanning a subject using an X-ray beam having a plurality of X-rays emitted from a tube through a collimator, the tilt image scan method comprising:
acquiring reconstruction parameters of a target tilt image;
determining minimum beam widths of the X-rays to be emitted from the tube at each angle based on the reconstruction parameters; and
controlling the collimator at each angle where the tube locates so that a beam width of the X-ray beam after the X-ray beam passes through the collimator is equal to the corresponding minimum beam width, thereby scanning the subject.

2. The tilt image scan method of claim 1, wherein acquiring reconstruction parameters comprises:
scanning the subject to obtain 90 degree and 0 degree location images; and
determining the reconstruction parameters of the target tilt image based on the location images.

3. The tilt image scan method of claim 1, wherein the reconstruction parameters include position, angle, layer thickness and size of reconstruction field of view.

4. The tilt image scan method of claim 3, wherein determining a minimum beam width comprises:
obtaining a layer thickness of a non-tilt tomographic image based on the position, angle, the thickness, and a required resolution;
obtaining a scan range of the non-tilt tomographic images necessary for reconstructing the target tilt image based on the layer thickness and the angle;
obtaining reconstruction areas on each slice of the non-tilt tomographic images for reconstructing the target tilt image based on the position and the angle of the target tilt image; and
determining the minimum beam widths of the tube at each angle based on the reconstruction areas.

5. A tilt image reconstruction method for use in scanning a subject by an X-ray beam having a plurality of X-rays emitted from a tube through a collimator, the tilt image reconstruction method comprising:
acquiring reconstruction parameters of a target tilt image;
determining minimum beam widths of the X-rays to be emitted from the tube at each angle based on the reconstruction parameters;
controlling the collimator at each angle where the tube locates, so that a beam width of the X-ray beam after the X-ray beam passes through the collimator is equal to the corresponding minimum beam width, thereby scanning the subject; and
reconstructing the target tilt image based on data obtained from the scan.

6. The tilt image reconstruction method of claim 5, wherein acquiring reconstruction parameters comprises:
scanning the subject to obtain 90 degree and 0 degree location images;
determining the reconstruction parameters of the target tilt image based on the location images.

7. The tilt image reconstruction method of claim 6, wherein the reconstruction parameters include position, angle, layer thickness and size of reconstruction field of view.

8. The tilt image reconstruction method of claim 7, wherein determining minimum beam widths comprises:
obtaining the layer thickness of a non-tilt tomographic image based on the position, the angle, the thickness, and a required resolution;
obtaining a scan range of non-tilt tomographic images necessary for reconstructing the target tilt image based on the layer thickness and the angle;
obtaining reconstruction areas on each slice of the non-tilt tomographic images for reconstructing the target tilt image based on the position and the angle of the target tilt image;
determining the minimum beam widths of the tube at each angle based on the reconstruction areas.

9. The tilt image reconstruction method of claim 5, wherein reconstructing the target tilt image comprises reconstructing by a local reconstruction technique or reconstructing by filtered back-projection.

10. A tilt image scan apparatus comprising:
an X-ray tube configured to emit an X-ray beam that includes a plurality of X-rays;
a collimator through which the X-ray beam passes;
an acquisition unit configured to acquire reconstruction parameters of a target tilt image;
a first unit for determining configured to determine minimum beam widths of the X-rays to be emitted from the tube at each angle; and
a control unit configured to control the collimator at each angle where the tube locates so that a beam width after the X-ray beam passes through the collimator is equal to the corresponding minimum beam width, thereby scanning the subject.

11. The tilt image scan apparatus of claim 10, further comprising a reconstruction unit configured to reconstruct the target tilt image based on data obtained from the scan.

12. The tilt image scan apparatus of claim 11, wherein the acquisition unit comprises:
a scanning unit configured to scan the subject to obtain 90 degree and 0 degree location images;
a second unit for determining, the reconstruction parameters of the target tilt image based on the location images.

13. The tilt image scan apparatus of claim 10, wherein the reconstruction parameters include position, angle, layer thickness, and size of reconstruction field of view.

14. The tilt image scan apparatus of claim 13, wherein the first unit for determining comprises:
a first unit for obtaining configured to determine a layer thickness of a non-tilt tomographic image based on the position, the angle, the thickness, and a required resolution;
a second unit for obtaining configured to:
determine a scan range of the non-tilt tomographic images necessary for reconstructing the target tilt image based on the layer thickness and the angle; and
determine reconstruction areas on each slice of the non-tilt tomographic images for reconstructing the target tilt image based on the position and the angle of the target tilt image; and
a third unit for determining configured to determine the minimum beam widths of the tube at each angle based on the reconstruction areas.

15. The tilt image scan apparatus of claim 14, wherein the reconstruction unit comprises a unit for reconstructing by a local reconstruction technique or reconstructing by filtered back-projection.

16. The tilt image scan method of claim 2, wherein the reconstruction parameters include position, angle, layer thickness, and size of reconstruction field of view.

17. The tilt image reconstruction method of claim 6, wherein reconstructing the target tilt image comprises reconstructing by a local reconstruction technique or reconstructing by filtered back-projection.

18. The tilt image reconstruction method of claim 7, wherein reconstructing the target tilt image comprises reconstructing by a local reconstruction technique or reconstructing by filtered back-projection.

19. The tilt image scan apparatus of claim 11, wherein the reconstruction parameters include position, angle, layer thickness, and size of reconstruction field of view.

20. The tilt image scan apparatus of claim 12, wherein the reconstruction parameters include position, angle, layer thickness, and size of reconstruction field of view.
